(19) United States
(12) Patent Application Publication
(10) Pub. No.: US 2019/0357888 A1
(43) Pub. Date: Nov. 28, 2019

(54) ULTRASOUND OBSERVATION APPARATUS AND OPERATION METHOD OF ULTRASOUND OBSERVATION APPARATUS

(71) Applicant: OLYMPUS CORPORATION, Tokyo (JP)

(72) Inventor: Tatsuya MIYAKE, Tokyo (JP)

(73) Assignee: OLYMPUS CORPORATION, Tokyo (JP)

(21) Appl. No.: 16/532,975

(22) Filed: Aug. 6, 2019

Related U.S. Application Data
(63) Continuation of application No. PCT/JP2018/005837, filed on Feb. 20, 2018.

(30) Foreign Application Priority Data
Mar. 6, 2017 (JP) 2017-041823

(51) Int. Cl. A61B 8/08; A61B 8/14; A61B 8/00; G06T 7/00; G06K 9/62; A61B 8/12

(52) U.S. Cl. CPC A61B 8/485; A61B 8/14; A61B 8/469; G06T 7/0014; G06K 2009/6213; A61B 8/12; G06T 2207/10132; G06T 2207/30004; G06T 2207/10068; G06K 9/6212

(57) ABSTRACT

An ultrasound observation apparatus includes: an agreement determination circuit configured to compare a reference image that is an ultrasound image chosen from ultrasound images and in which at least one region of interest is set and a latest ultrasound image with each other at least partly, and determine whether the reference image and the latest ultrasound image agree with each other; and a measurement circuit configured to, when the agreement determination circuit determines that the reference image and the latest ultrasound image agree with each other, transmit a push pulse to the at least one region of interest to cause shear waves, transmit and receive a tracking pulse to detect propagation of the shear waves, and measure elasticity characteristics in the at least one region of interest.

### CROSS-REFERENCE TO RELATED APPLICATION

This application is a continuation of PCT International Application No. PCT/JP2018/005837, filed on Feb. 20, 2018, which designates the United States, incorporated herein by reference, and which claims the benefit of priority from Japanese Patent Application No. 2017-041823, filed on Mar. 6, 2017, incorporated herein by reference.

### BACKGROUND

### 1. Technical Field

The present disclosure relates to an ultrasound observation apparatus, and an operation method of an ultrasound observation apparatus.

### 2. Related Art

In the field of medicine, ultrasound observation apparatuses that generate ultrasound images based on ultrasound signals obtained by an ultrasound transducer by transmitting and receiving the ultrasound signals to and from a subject to be observed have been used.

Some ultrasound observation apparatuses set a region of interest in an ultrasound image, transmit a push pulse to the region of interest to cause shear waves, transmit and receive a tracking pulse to detect propagation of the shear waves, and accurately measure elasticity characteristics of the region of interest (refer to, for example, Japanese Laid-open Patent Publication No. 2016-67392).

### SUMMARY

In some embodiments, provided is an ultrasound observation apparatus configured to generate a plurality of ultrasound images based on echo signals that are electric signals into which ultrasound echoes that are ultrasound transmitted to a subject to be observed and then reflected from the subject to be observed are converted. The ultrasound observation apparatus includes: an agreement determination circuit configured to compare a reference image that is an ultrasound image chosen from the ultrasound images and in which at least one region of interest is set and a latest ultrasound image with each other at least partly, and determine whether the reference image and the latest ultrasound image agree with each other; and a measurement circuit configured to, when the agreement determination circuit determines that the reference image and the latest ultrasound image agree with each other, transmit a push pulse to the at least one region of interest to cause shear waves, transmit and receive a tracking pulse to detect propagation of the shear waves, and measure elasticity characteristics in the at least one region of interest.

In some embodiments, provided is an operation method of an ultrasound observation apparatus configured to generate a plurality of ultrasound images based on echo signals that are electric signals into which ultrasound echoes that are ultrasound transmitted to a subject to be observed and then reflected from the subject to be observed are converted. The method includes: by an agreement determination circuit, comparing a reference image that is an ultrasound image chosen from the ultrasound images and in which at least one region of interest is set and a latest ultrasound image with each other at least partly and determining whether the reference image and the latest ultrasound image agree with each other; and by a measurement circuit, when the agreement determination circuit determines that the reference image and the latest ultrasound image agree with each other, transmitting a push pulse to the at least one region of interest to cause shear waves, transmitting and receiving a tracking pulse to detect propagation of the shear waves, and measuring elasticity characteristics in the at least one region of interest.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a block diagram illustrating a configuration of an ultrasound diagnostic system including an ultrasound observation apparatus according to a first embodiment of the present disclosure;

FIG. 2 is a flowchart illustrating an overview of a process performed by the ultrasound observation apparatus according to the first embodiment of the present disclosure;

FIG. 3 is a diagram illustrating comparison between a reference image and the latest ultrasound image;

FIG. 4 is a flowchart illustrating an overview of a process performed by an ultrasound observation apparatus according to a second embodiment of the present disclosure; and

FIG. 5 is a diagram illustrating that a plurality of regions of interest are set in a reference image.

### DETAILED DESCRIPTION

Embodiments of an ultrasound observation apparatus, an operation method of an ultrasound observation apparatus; and an operation program for an ultrasound observation apparatus according to the present disclosure will be described with reference to the drawings. The embodiments do not limit the disclosure. The disclosure is generally applicable to an ultrasound observation apparatus capable of measuring elasticity characteristics using shear waves, an operation method of an ultrasound observation apparatus, and an operation program for an ultrasound observation apparatus.

In the illustration of the drawings, like or corresponding components are denoted with like reference numerals as appropriate. Furthermore, it should be noted that the drawings are schematic and thus the relationship in size among components and the ratio among components can differ from actual ones. The drawings may also contain parts whose relationship in size or ratio differ among the drawings.

### First Embodiment

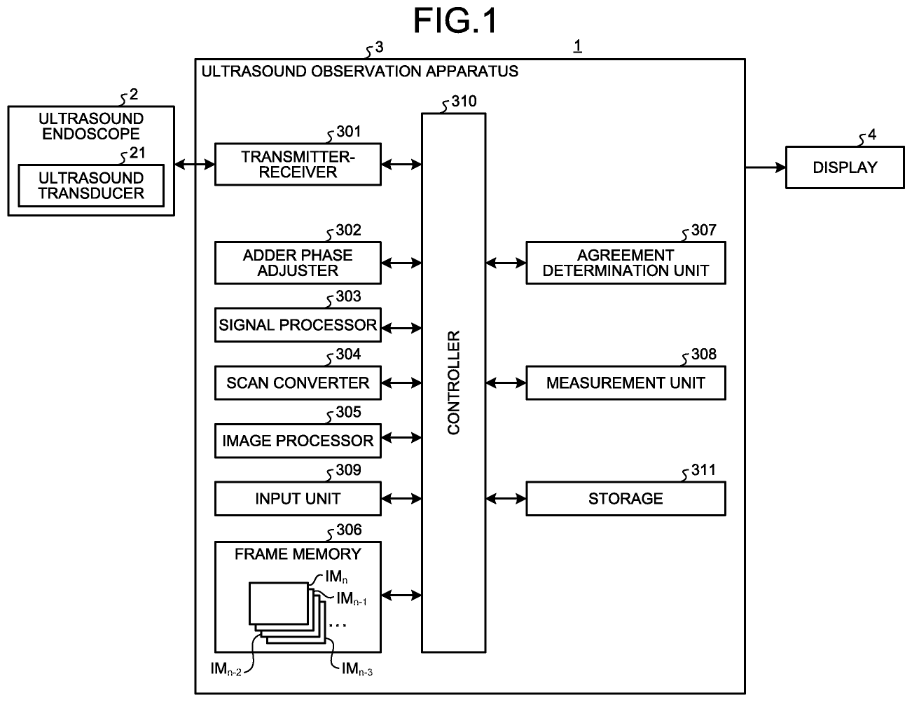

FIG. 1 is a block diagram illustrating a configuration of an ultrasound diagnostic system including an ultrasound observation apparatus according to a first embodiment of the disclosure. An ultrasound diagnostic system 1 illustrated in FIG. 1 includes an ultrasound endoscope 2 that transmits ultrasound to a subject to be observed and receives the ultrasound reflected from the subject; an ultrasound observation apparatus 3 that generates an ultrasound image based on an ultrasound signal that is acquired by the ultrasound endoscope 2; and a display 4 that displays the ultrasound image that is generated by the ultrasound observation apparatus 3.

The ultrasound endoscope 2 includes an ultrasound transducer 21 that converts an electric signal that is input from the ultrasound observation apparatus 3 into an ultrasound pulse (acoustic pulse) and applies the ultrasound pulse onto a subject and converts ultrasound echo that is reflected from the subject into an electric echo signal representing the ultrasound echo by a change in voltage and outputs the echo signal.

Under the control of the ultrasound observation apparatus 3, the ultrasound transducer 21 transmits a push pulse that is focused on a given focal point to the subject. Under the control of the ultrasound observation apparatus 3, the ultrasound transducer 21 further transmits and receives a tracking pulse to detect propagation of shear waves that are caused by the push pulse.

The ultrasound transducer 21 is arranged at the head of an insertion part to be inserted into the subject. The ultrasound transducer 21 may be any one of a convex-type ultrasound transducer and a radial-type ultrasound transducer. A plurality of elements serving as the ultrasound transducer 21 are provided in an array and, by electrically switching the elements relating to transmission and reception and delaying the transmission and reception at each of the elements, the ultrasound endoscope 2 causes electric scanning.

The ultrasound endoscope 2 normally includes an imaging optical system and an imaging device and the ultrasound endoscope 2 is inserted into a digestive tract of the subject (the esophagus, the stomach, the duodenum or the large intestine) or a respiratory organ (the trachea or a bronchi), which makes it possible to capture images of the digestive tract or the respiratory organ and their surrounding organs (the pancreas, the gallbladder, the bile duct, the bile tract, lymph nodes, mediastinal organs, blood vessels, etc.). The ultrasound endoscope 2 includes a light guide that guides illumination light that is applied to the subject to capture an image. While the top of the light guide reaches the top of the insertion part to be inserted into the subject, the proximal end of the light guide is connected to a light source device that generates the illumination light.

The ultrasound observation apparatus 3 includes a transmitter-receiver 301, an adder phase adjuster 302, a signal processor 303, a scan converter 304, an image processor 305, a frame memory 306, an agreement determination unit 307, a measurement unit 308, an input unit 309, a controller 310 and a storage 311.

The transmitter-receiver 301 is electrically connected to the ultrasound endoscope 2 and transmits a transmission signal based on a given waveform and given transmission timing and receives an echo signal that is an electric reception signal from the ultrasound transducer 21. Furthermore, the transmitter-receiver 301 transmits a push pulse and transmits and receives a tracking pulse.

The transmitter-receiver 301 also has a function of transmitting various control signals that are output from the controller 310 to the ultrasound endoscope 2, receiving various types of information containing an identification ID from the ultrasound endoscope 2, and transmitting the various types of information to the controller 310.

The adder phase adjuster 302 receives the echo signal from the transmitter-receiver 301 and generates and outputs data of a digital high-frequency (radio frequency (RF)) signal (RF data below). The adder phase adjuster 302 performs sensitivity time control (STC) correction in which an echo signal with a deeper hydrophone depth is amplified at a higher amplification rate and, after performing a process, such as filtering, on the amplified echo signal, performs A/D conversion to generate time-domain RF data and outputs the RF data to the signal processor 303. The adder phase adjuster 302 includes a multi-channel circuit for beam synthetization corresponding to the elements that are arranged in an array.

The signal processor 303 generates digital B-mode reception data based on the RF data that is received from the transmitter-receiver 301. The signal processor 303 performs known processing, such as band-pass filtering, envelope detection and logarithmic transformation, on the RF data to generate digital B-mode reception data. In logarithmic transformation, a common logarithm that is an amount obtained by dividing the RD data by a reference voltage Vc is expressed by a decibel value. The signal processor 303 outputs the generated B-mode reception data to the image processor 305. The signal processor 303 is achieved using a central processing unit (CPU) and various types of operational circuits.

The scan converter 304 converts a scan direction for the B-mode reception data that is received from the signal processor 303 and generates frame data. Specifically, the scan converter 304 converts the scan direction for the B-mode reception data from the ultrasound scan direction to the display direction of the display 4.

The image processor 305 generates B-mode image data (also simply referred to as image data below) containing an ultrasound image that is a B-mode image that is obtained by converting the amplitude of the echo signal into an illuminance and that is displayed. The image processor 305 generates the B-mode image data by performing signal processing using a known technology, such as gain processing, contrast processing, etc., on the frame data from the scan converter 304 and interpolation, etc., on the data corresponding to the data step width that is determined according to the image display range in the display 4. The B-mode image is a grayscale image where the R (red), G (green), and B (blue) values that are variables in the case where the RGB coordinate system is employed as a color space are caused to agree with each other.

After performing coordinate transformation in which rearrangement is performed to enable spatially correct representation of a scanning area on the B-mode reception data from the signal processor 303, the image processor 305 infills the B-mode reception data by performing decimation processing in the B-mode reception data, thereby generating the B-mode image data.

Furthermore, the image processor 305 generates an elasticity image obtained by superimposing elasticity characteristics of the region of interest on a reference image to be described below. Specifically, the image processor 305 generates the elasticity image by superimposing the elasticity characteristics of the region of interest on the reference image by coloring the region of interest according to a value of elasticity. Note that the image processor 305 may generate an image obtained by superimposing the elasticity characteristics of the region of interest as a numerical value. The image processor 305 is achieved using a CPU and various operational circuits.

The frame memory 306 is achieved using, for example, a ring buffer and stores a certain number of ultrasound images (a given number of frames N: N=n, n−1, n−2, n−3, . .) in a chronological order. When the space is insufficient (when B-mode image data of the given number of frames is stored), the oldest B-mode image data is overwritten with the latest B-mode image data to store the given number of the latest ultrasound images in the chronological order. As illustrated in FIG. 1, the frame memory 306 stores a plurality of ultrasound images (IMₙ₋₁IM_{N-2}, IM_{N-3}, . . . ) of the given number of frames ahead of the ultrasound image IMₙ of the n-th frame (n is a natural number equal to or larger than 2) that is the latest ultrasound image.

The agreement determination unit 307 compares a reference image that is an ultrasound image chosen from the ultrasound images stored in the frame memory 306 and in which a region of interest is set and the latest ultrasound image IMₙwith each other at least partly and determines whether the reference image and the latest ultrasound image IMₙ , agree with each other. Specifically, the agreement determination unit 307 compares the entire reference image and the entire ultrasound image IMₙ with each other with respect to the entire image based on, for example, at least any one of pattern matching, a statistical value that is calculated from illuminance histogram, and a difference in illuminance, thereby determining whether the reference image and the latest ultrasound image IMₙ agree with each other. The agreement determination unit 307 may determine agreement by comparing the reference image and the latest ultrasound image with each other with respect to the region of interest or the region of interest and the surroundings of the region of interest. The agreement determination unit 307 is achieved using a CPU and various operational circuits.

When the agreement determination unit 307 determines that the reference image and the latest ultrasound image IMₙ agree with each other, the measurement unit 308 transmits a push pulse to the region of interest to cause shear waves, transmits and receives a tracking pulse to detect propagation of the shear waves, and measures elasticity characteristics in the region of interest. The measurement unit 308 is achieved using a CPU and various types of processing circuits.

The input unit 309 is achieved with user interfaces, such as a keyboard, a mouse, and a touch panel, and receives input of various types of information. The input unit 309 receives an input of a freeze instruction signal that is an instruction input to cause the display 4 to display a frozen image. The input unit 309 receives an instruction input of the user to set a region of interest in a frozen image.

The controller 310 controls the entire ultrasound diagnostic system 1. The controller 310 is achieved using a CPU with operational and control functions and various types of operational circuits. The controller 310 reads information that is stored in the storage 311 from the storage 311 and overall controls the ultrasound observation apparatus 3 by executing various types of operational processing relating to the operation method of the ultrasound observation apparatus 3. Note that the controller 310 may be formed of the CPU that is shared among the signal processor 303, the image processor 305, the agreement determination unit 307, and the measurement unit 308.

The storage 311 stores various types of programs containing an operation program for executing the operation method of the ultrasound observation apparatus 3. The operation program is also recordable in a computer-readable recording medium, such as a hard disk, a flash memory, a CD-ROM, a DVD-ROM or a flexible disk, and thus is widely distributable. By downloading the various programs via a communication network, it is possible to acquire the above-described various programs. The communication network herein is achieved with, for example, an existing public network, a local area network (LAN), a wide area network (WAN), or the like and it does not matter whether the communication network is wired or wireless.

The storage 311 having the above-described configuration is achieved using a read only memory (ROM) in which the various programs are installed in advance, a random access memory (RAM) that stores operational processing parameters and data for each set of processing, etc.

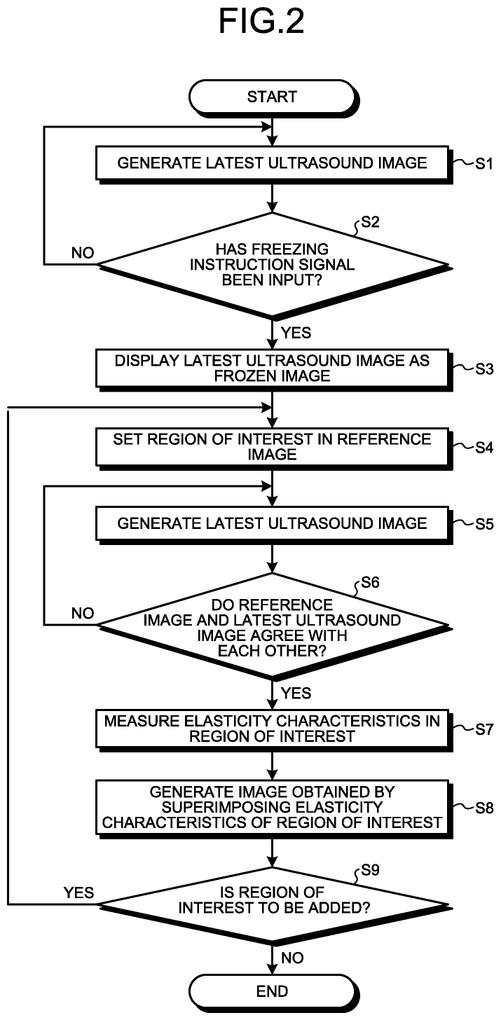

FIG. 2 is a flowchart of an overview of a process performed by the ultrasound observation apparatus according to the first embodiment of the present disclosure. As illustrated in FIG. 2, the image processor 305 performs image processing on the B-mode reception data from the signal processor 303 to generate the latest ultrasound image IMₙ (step S1). The ultrasound observation apparatus 3 causes the display 4 to display the latest ultrasound image IMₙ as appropriate. In other words, the display 4 displays the latest ultrasound image IMₙ as a live display.

The controller 310 determines whether a freezing instruction signal has been input to the input unit 309 (step S2). When the controller 310 determines that the freezing instruction signal has not been input (NO at step S2), the process of step S1 and step S2 is executed repeatedly.

When the controller 310 determines that the freezing instruction signal has been input (YES at step S2), the ultrasound observation apparatus 3 causes the display 4 to display the latest ultrasound image IMₙ at that time as a frozen image (step S3). When the ultrasound observation apparatus 3 having a pre-freezing function is used, the display 4 may be caused to display, as a frozen image, an ultrasound image with less blur that is chosen by the pre-freezing function from the ultrasound images that are stored in the frame memory 306.

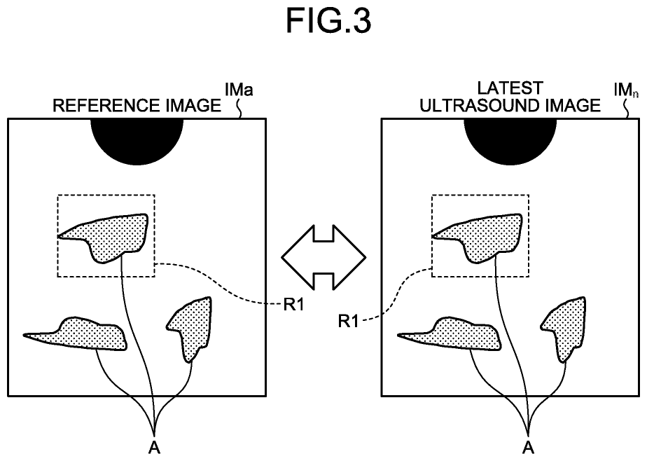

The user then chooses a reference image from the ultrasound images stored in the frame memory 306 and sets a region of interest in the reference image (step S4). FIG. 3 is a diagram illustrating comparison between the reference image and the latest ultrasound image. As illustrated in FIG. 3, the user sets a region of interest R1 such that one of a plurality of subjects A to be observed, such as lesions, in the reference image IMa is surrounded. Specifically, the user makes a given instruction input from the input unit 309 and sets a desired area in the reference image IMa as the region of interest R1.

When setting the region of interest R1 completes, the image processor 305 performs image processing on the B-mode reception data from the signal processor 303 as at step S1 to generate the latest ultrasound image IMₙ (step S5). In other words, the display on the display 4 is switched from a frozen display to a live display.

The agreement determination unit 307 determines whether the reference image IMa and the latest ultrasound image IMₙ agree with each other (step S6). The agreement determination unit 307, for example, performs pattern matching on the reference image IMa and the latest ultrasound image IMₙ with respect to the entire image and, when the calculated similarity is at or above a threshold, determines that the reference image IMa and the latest ultrasound image IMₙ agree with each other. When the agreement determination unit 307 determines that the reference image IMa and the latest ultrasound image IMₙ do not agree with each other (NO at step S6), the process of step S5 and S6 is repeatedly executed. When a given instruction input of an instruction to end is input during repetition of the process of step S5 and step S6, the process series ends promptly. When the time during which the process of steps S5 and S6 is repeated exceeds a given time, the threshold of similarity to determine agreement between the reference image IMa and the latest ultrasound image IMₙ may be lowered or a given alert may be made with, an alarm, or the like.

When the agreement determination unit 307 determines that the reference image IMa and the latest ultrasound image IMₙ agree with each other (YES at step S6), the measurement unit 308 transmits a push pulse to the region of interest R1 to cause shear waves, transmits and receives a tracking pulse to detect propagation of the shear waves, and measures elasticity characteristics in the region of interest R1 (step S7). The image processor 305 accordingly generates an image obtained by superimposing the elasticity characteristics of the region of interest R1 on the reference image IMa (step S8). The ultrasound observation apparatus 3 then causes the display 4 to display, as a frozen display, the image obtained by superimposing the elasticity characteristics of the region of interest R1 on the reference image IMa by color or numeric value. Note that the image processor 305 may generate an image obtained by superimposing the elasticity characteristics of the region of interest R1 on the latest ultrasound image IMₙ.

The controller 310 then determines whether an instruction to add a region of interest has been input to the input unit 309 (step S9). When the controller 310 determines that the instruction to add a region of interest has been input to the input unit 309 (YES at step S9), the controller 310 returns to step S4 to measure elasticity characteristics of an added region of interest. In this case, at step S8, the image processor 305 generates an image obtained by superimposing both sets of elasticity characteristics of the pre-set region of interest and the added region of interest on the reference image IMa. When the second and following measurement is performed, it is preferable that measurement be performed after a given cooling period (for example, few seconds) passes.

When the controller 310 determines that the instruction to add a region of interest has not been input to the input unit 309 (NO at step S9), the process series ends according an input of the given instruction to end.

As described above, according to the first embodiment, when the agreement determination unit 307 determines that the reference image IMa and the latest ultrasound image IMₙ agree with each other, the measurement unit 308 automatically measures elasticity characteristics in a region of interest. As a result, when the ultrasound transducer 21 is at the same position as that where the ultrasound transducer 21 captures the reference image IMa in a living body, the measurement unit 308 measures the elasticity characteristics in the region of interest and thus, when observation using shear waves is performed, even if it is difficult to maintain a relative position between the living body and the ultrasound transducer, the elasticity characteristics in the region of interest can be measured accurately.

### Second Embodiment

An ultrasound observation apparatus according to a second embodiment differs from the first embodiment in the process performed by the ultrasound observation apparatus but has the same configuration as that of the first embodiment and thus description thereof will be omitted as appropriate. Also as for the same process as that of the first embodiment, description thereof will be omitted as appropriate.

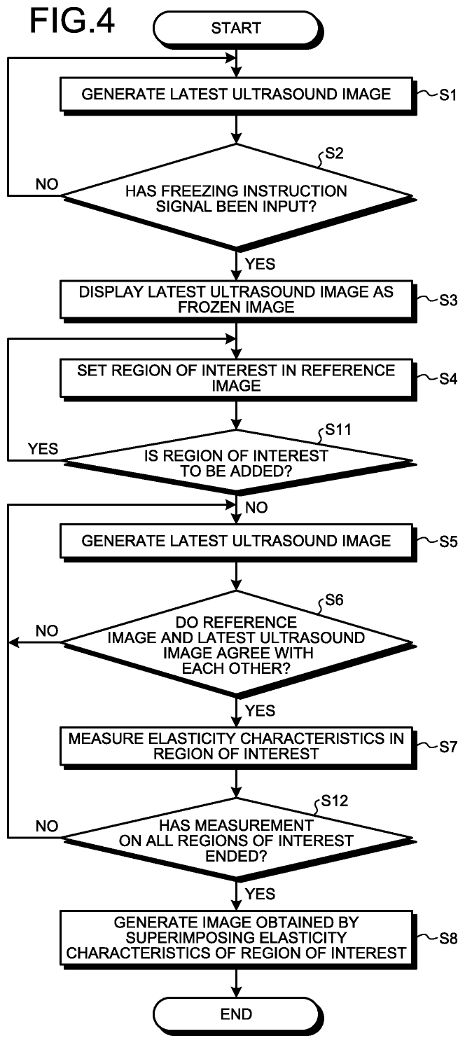

FIG. 4 is a flowchart illustrating an overview of the process performed by the ultrasound observation apparatus according to the second embodiment of the present disclosure. As illustrated in FIG. 4, first of all, the process of steps S1 to S4 is performed as in the first embodiment.

The controller 310 subsequently determines whether a region of interest is to be added (step S11). Specifically, for example, the display 4 is caused to display dialog boxes to choose whether a region of interest is added and the controller 310 determines which of the dialog boxes the user chooses.

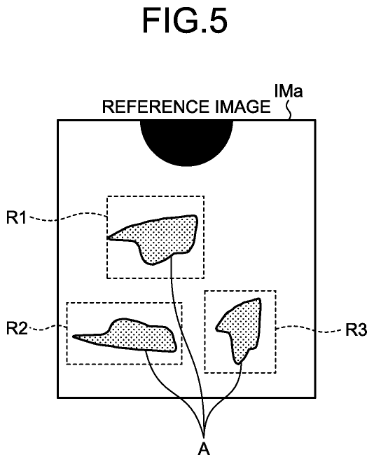

When the controller 310 determines that a region of interest is to be added (YES at step S11), the user sets a region of interest to be added to the reference image (step S4). FIG. 5 is a diagram illustrating that a plurality of regions of interest are set in a reference image. As illustrated in FIG. 5, the user sets regions of interests R1 to R3 such that a plurality of subjects A to be observed in the reference image IMa are respectively surrounded. When elasticity characteristics are measured using shear waves, the size of a region of interest that can be set can be limited because of attenuation of shear waves. For this reason, when there are a plurality of subjects A to be observed, it is necessary to set a plurality of regions of interest such that the subjects A to be observed are respectively surrounded.

When the controller 310 determines that no region of interest is to be added (NO at step S11), as at step S1, the image processor 305 performs image processing on B-mode reception data from the signal processor 303 to generate the latest ultrasound image IMₙ (step S5).

The agreement determination unit 307 determines whether the reference image IMa and the latest ultrasound image IMₙ agree (step S6). When the agreement determination unit 307 determines that the reference image IMa and the latest ultrasound image IMₙ do not agree with each other (NO at step S6), the process of steps S5 and S6 is repeatedly executed.

When the agreement determination unit 307 determines that the reference image IMa and the latest ultrasound image IMₙ agree with each other (YES at step S6), the measurement unit 308 transmits a push pulse to the region of interest R1 to cause shear waves, transmits and receives a tracking pulse to detect propagation of the shear waves, and measures elasticity characteristics in the region of interest R1 (step S7).

The controller 310 then determines whether measurement on all the regions of interest has ended (step S12). When the controller 310 determines that measurement on all the regions of interest has not ended (NO at step S12), the controller 310 returns to step S5 to perform measurement on the next region of interest. In other words, each time the agreement determination unit 307 determines that the reference image IMa and the latest ultrasound image IMₙ agree with each other, the measurement unit 308 repeatedly performs measurement of elasticity characteristics of a different region of interest to measure elasticity characteristics of all the regions of interest. When the second or following measurement is performed, it is preferable to perform measurement after a given cooling period (for example, few seconds) passes. When measurement of elasticity characteristics of each region of interest is started, a notification of the start of measurement may be made by display on the display 4 or sound. Furthermore, on which region of interest measurement is performed may be displayed on the display 4.

When the controller 310 determines that measurement on all the regions of interest has ended (YES at step S12), the image processor 305 generates an image obtained by superimposing the elasticity characteristics of the regions of interest (for example, the regions of interest R1 to R3 illustrated in FIG. 5) on the reference image IMa (step S8). The ultrasound observation apparatus 3 then causes the display 4 to display as a frozen display the image obtained by superimposing the elasticity characteristics of the regions of interest R1 to R3 on the reference image IMa. Note that the image processor 305 may generate an image obtained by superimposing the elasticity characteristics of the regions of interest R1 to R3 on the latest ultrasound image IMₙ. In response to a given input of an instruction to end, the process series then ends. Note that each time measurement of elasticity characteristics of the regions of interest R1 to R3 ends, the ultrasound observation apparatus 3 may cause the display 4 to display as a frozen image the image obtained by superimposing the elasticity characteristics of the regions of interest on which measurement has ended on the reference image IMa.

As described above, according to the second embodiment, when it is determined that the agreement determination unit 307 determines that the reference image IMa and the latest ultrasound image IMₙ agree with each other, elasticity characteristics in the regions of interest are automatically calculated sequentially. As a result, when performing measurement using shear waves, even in the case where a plurality regions of interest have been set and thus it is difficult to keep maintaining relative positions between each living body and the ultrasound transducer, it is possible to accurately measure the elasticity characteristics in the regions of interest.

For the above-described embodiments, the examples where the ultrasound endoscope 2 is applied to the ultrasound observation apparatus 3 according to the present disclosure have been described. Alternatively, an external ultrasound probe that emits ultrasound pulses from the body surface of a subject may be used.

According to the disclosure, it is possible to achieve an ultrasound observation apparatus, an operation method of an ultrasound observation apparatus, and an operation program for an ultrasound observation apparatus that, when observation using shear waves is performed, enable accurate measurement of elasticity characteristics in a region of interest even if it is difficult to maintain a relative position between a living body and an ultrasound transducer.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasound observation apparatus configured to generate a plurality of ultrasound images based on echo signals that are electric signals into which ultrasound echoes that are ultrasound transmitted to a subject to be observed and then reflected from the subject to be observed are converted, the ultrasound observation apparatus comprising:
an agreement determination circuit configured to
compare a reference image that is an ultrasound image chosen from the ultrasound images and in which at least one region of interest is set and a latest ultrasound image with each other at least partly, and
determine whether the reference image and the latest ultrasound image agree with each other; and
a measurement circuit configured to
when the agreement determination circuit determines that the reference image and the latest ultrasound image agree with each other, transmit a push pulse to the at least one region of interest to cause shear waves,
transmit and receive a tracking pulse to detect propagation of the shear waves, and
measure elasticity characteristics in the at least one region of interest.

2. The ultrasound observation apparatus according to claim 1, wherein the measurement circuit is configured to, when the region of interest is a plurality of regions of interest, each time the agreement determination circuit determines that the reference image and the latest ultrasound image agree with each other, repeatedly measure elasticity characteristics of a different region of interest to measure elasticity characteristics of all the regions of interest.

3. The ultrasound observation apparatus according to claim 1, wherein the agreement determination circuit is configured to determine whether the reference image and the latest ultrasound image agree with each other, based on at least any one of pattern matching, a statistical value that is calculated from a histogram of illuminance, and an amount of difference in illuminance.

4. The ultrasound observation apparatus according to claim 1, wherein the agreement determination circuit is configured to compare the reference image and the latest ultrasound image with each other with respect to at least any one of the entire image, the at least one region of interest, and the at least one region of interest and surroundings of the at least one region of interest and determine whether the reference image and the latest ultrasound image agree with each other.

5. The ultrasound observation apparatus according to claim 1, wherein the ultrasound image is an image that is captured by an ultrasound endoscope in which an ultrasound transducer that transmits and receives ultrasound is arranged at a head of an insertion part to be inserted into a subject.

6. An operation method of an ultrasound observation apparatus configured to generate a plurality of ultrasound images based on echo signals that are electric signals into which ultrasound echoes that are ultrasound transmitted to a subject to be observed and then reflected from the subject to be observed are converted, the method comprising:
by an agreement determination circuit, comparing a reference image that is an ultrasound image chosen from the ultrasound images and in which at least one region of interest is set and a latest ultrasound image with each other at least partly and determining whether the reference image and the latest ultrasound image agree with each other; and
by a measurement circuit, when the agreement determination circuit determines that the reference image and the latest ultrasound image agree with each other, transmitting a push pulse to the at least one region of interest to cause shear waves, transmitting and receiving a tracking pulse to detect propagation of the shear waves, and measuring elasticity characteristics in the at least one region of interest.
